(19) United States
(12) Patent Application Publication
(10) Pub. No.: US 2026/0283846 A1
(43) Pub. Date: Sep. 24, 2026

(54) AN OPHTHALMIC SURGERY INSTRUMENT

(71) Applicant: D.O.R.C. Dutch Ophthalmic Research Center (International) B.V., Zuidland (NL)

(72) Inventor: Nadieh BERGWERF, Zwijndrecht (NL)

(21) Appl. No.: 19/473,606

(22) PCT Filed: Apr. 11, 2024
(86) PCT No.: PCT/NL2024/050186

§ 371 Date: Oct. 8, 2025

(30) Foreign Application Priority Data
Apr. 12, 2023 (NL) 2034560

(51) Int. Cl. A61F 9/007

(52) U.S. Cl. CPC A61F 9/007

(57) ABSTRACT

The invention relates to an ophthalmic surgery instrument, comprising a handling unit, a surgical module provided on the handling unit, and a shaft at least partially surrounding the surgical module, wherein the surgical module and the shaft are mutually movable relative. The handling unit further comprises a guiding element arranged at a distal end of the handling unit. The handling unit comprises an actuation unit having a plurality of actuation arms, and a driving unit for driving the shaft relative to the guiding element back and forth through the central bore of the guiding element, wherein the driving unit includes an annular element, at a proximal side of the guiding element, to which annular element distal ends of the actuation arms are pivotably connected for moving the annular element towards the guiding element.

### SUMMARY OF THE INVENTION

The invention relates to an ophthalmic surgery instrument, comprising a handling unit having a longitudinal axis, a surgical module provided on the handling unit and extending away therefrom along the longitudinal axis thereof, and a shaft at least partially surrounding the surgical module, wherein the surgical module and the shaft are mutually movable relative along the longitudinal axis of the handling unit, a guiding element arranged at a distal end of the handling unit, the guiding element being provided with a central bore that is traversed by the surgical module and the shaft extending from the handling unit outwardly along the longitudinal axis thereof, wherein the handling unit comprises an actuation unit comprising a plurality of actuation arms arranged around the longitudinal axis of the handling unit, the actuation arms each having a proximal end and a distal end, and a driving unit arranged to, upon manual actuation of the actuation unit, drive the shaft relative to the guiding element back and forth through the central bore of the guiding element.

The above ophthalmic surgery instrument is known for example for ophthalmic surgery in the interior of the eye wherein the surgical module includes a movable distal portion such as a forceps. By providing a shaft partially surrounding the surgical module such that the shaft and the surgical module are movable relative to each other, the movable distal portion can selectively at least partly be covered or closed, or exposed or opened by the shaft, e.g. for actuating the surgical module and/or for inserting or removing the surgical module from the eye.

There is an ongoing need to improve manual operation of the ophthalmic surgery instrument.

It is an object of the present invention to provide an ophthalmic surgery instrument having improved manual operation characteristics. Therefore, according to the invention, an ophthalmic surgery instrument is provided, comprising a handling unit having a longitudinal axis, a surgical module provided on the handling unit and extending away therefrom along the longitudinal axis thereof, and a shaft at least partially surrounding the surgical module, wherein the surgical module and the shaft are mutually movable relative along the longitudinal axis of the handling unit, a guiding element arranged at a distal end of the handling unit, the guiding element being provided with a central bore that is traversed by the surgical module and the shaft extending from the handling unit outwardly along the longitudinal axis thereof, wherein the handling unit comprises an actuation unit comprising a plurality of actuation arms arranged around the longitudinal axis of the handling unit, the actuation arms each having a proximal end and a distal end, and a driving unit arranged to, upon manual actuation of the actuation unit, drive the shaft relative to the guiding element back and forth through the central bore of the guiding element, wherein the driving unit includes an annular element arranged at a proximal side of the guiding element so as to move, in association with the shaft, along the longitudinal axis, and wherein the number of actuation arms is at least three, and wherein the distal ends of the actuation arms are pivotably connected to the annular element such that upon compressing at least a subset of the actuation arms the annular element moves along the longitudinal axis towards the guiding element.

By providing the driving unit with an annular element arranged at a proximal side of the guiding element and by pivotably connecting the distal ends of the actuation arms to said annular element, so as to move, in association with the shaft, along the longitudinal axis towards the guiding element, operation of the instrument can be improved, e.g. in that operation of the shaft can be performed easily and controllably, based on a relatively simple and robust design.

Advantageously, the actuation arms and the annular element form a radial-to-axial linkage constructed and arranged to convert a radial movement provided by the actuation unit to an axial movement of the shaft, thereby realizing a robust implementation of the instrument.

Preferably, the annular element of the driving unit includes a flange radially extending from the longitudinal axis, wherein the distal ends of the actuation arms are pivotably connected to said flange, such that the actuation arms are mutually coupled. Alternatively, the distal ends of the actuation arms may be connected to another portion of the annular element, e.g. to corresponding radially extending members of said annular element.

The annular element may axially, preferably removably, coupled to the shaft for driving said shaft along the longitudinal axis.

Further advantageous embodiments according to the invention are described in the following claims.

It should be noted that the technical features described above or below may each on its own be embodied in an ophthalmic surgery instrument and/or in a method, i.e. isolated from the context in which it is described, separate from other features, or in combination with only a number of the other features described in the context in which it is disclosed. Each of these features may further be combined with any other feature disclosed, in any combination.

The invention will be further elucidated on the basis of exemplary embodiments which are represented in the drawings. The exemplary embodiments are given by way of non-limitative illustrations of the invention. In the drawings:

### BRIEF DESCRIPTION OF THE DRAWINGS

FIG. 1 shows a schematic side view of an ophthalmic surgery instrument according to the invention, and

FIG. 2 shows a schematic side view of the ophthalmic surgery instrument shown in FIG. 1, without actuation unit.

### DETAILED DESCRIPTION

In the figures identical or corresponding parts are represented with the same reference numerals. The drawings are only schematic representations of embodiments of the invention.

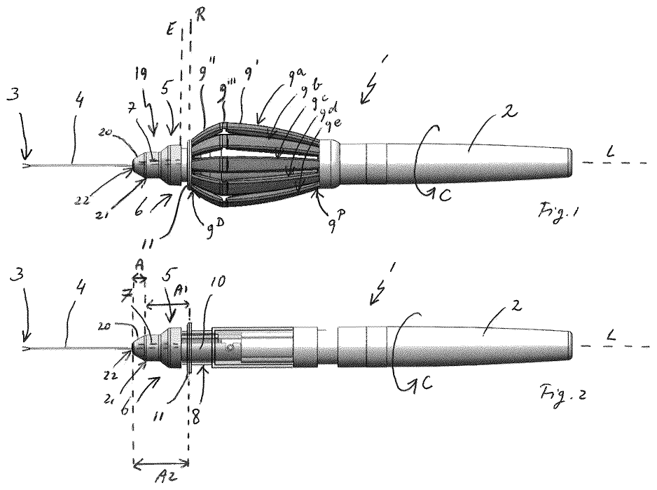

FIG. 1 shows a schematic side view of an ophthalmic surgery instrument 1 according to the invention. The instrument 1 includes a handling unit 2 having a longitudinal axis L. Further, the instrument 1 includes a surgical module 3 provided on the handling unit 2 and extending away therefrom along the longitudinal axis L thereof, and a shaft 4 that at least partially surrounds the surgical module 3. The surgical module 3 and the shaft 4 are mutually movable along the longitudinal axis L of the handling unit 2. In the shown embodiment, the shaft 4 moves along the longitudinal axis L relative to the handling unit 2, while the surgical module 3 remains mainly stationary along the longitudinal axis L relative to the handling unit 2.

The ophthalmic surgery instrument 1 also includes a guiding element 5 arranged at a distal end 6 of the handling unit 2, the guiding element 5 being provided with a central bore 7 that is traversed by the surgical module 3 and the shaft 4 extending from the handling unit 2 outwardly along the longitudinal axis L thereof.

The handling unit 2 further comprises an actuation unit 9 having a plurality of actuation arms 9a-e, also referred to as levers, arranged along the circumferential direction C around the longitudinal axis L of the handling unit 2. The actuation arms or levers 9, also referred to as basket, each have a proximal end 9^{P} and a distal end 9^{D}. Generally, the actuation arms 9 are bendable, and adjustable between a compressed state and a decompressed state, wherein the actuation arms 9 in the compressed state are closer to the longitudinal axis L of the handling unit 2 than in the decompressed state. Preferably, the actuation arms 9 are biased towards the decompressed state.

The handling unit 2 also comprises a driving unit 8 for driving, upon manual actuation of the actuation unit 9, the shaft 4 relative to the guiding element 5 back and forth through the central bore 7 of the guiding element 5.

FIG. 2 shows a schematic side view of the ophthalmic surgery instrument 1 shown in FIG. 1, without the actuation unit 9. In FIG. 2, the driving unit 8 is more visible than in FIG. 1. As shown, the driving unit 8 includes an annular element 10 arranged at a proximal side of the guiding element 5 so as to move, in association with the shaft 4, along the longitudinal axis L.

The distal ends 9^{D} of the actuation arms 9 are pivotably connected to the annular element 10 such that upon compressing at least a subset of the actuation arms 9 the annular element 10 moves along the longitudinal axis L towards the guiding element 5.

In the shown embodiment, the number of visible actuation arms 9 is five, while the total number of actuation arms 9 of the actuation unit 9, around the longitudinal axis L of the handling unit 2, in the circumferential direction C, is eight. The number of actuation arms 9 may be more or less than eight. Preferably, the number of actuation arms 9 is even, such as four, six or eight. Then, the circumferential positions of the individual actuation arms can be chosen, preferably evenly distributed in the circumferential direction C, in such a manner that each actuation arm forms, together with another actuation arm positioned at circa 180 degrees in the circumferential direction C, a pair of opposing actuation arms 9. The actuation arms 9 can be formed as pairs of actuation arms. A user of the ophthalmic surgery instrument 1 may operate a pair of actuation arms by pressing said specific pair of actuation arms simultaneously. The multiple pairs of opposing actuation arms each have a specific circumferential orientation with respect to the surgical module. As an example, in the case of eight actuation arms, the user may select a specific a specific circumferential orientation of the arms 9 with respect to the surgical module 3, e.g. circa 0 degrees, circa 45 degrees, circa 90 degrees or circa 135 degrees in the circumferential direction C. Upon compressing the selected pair of actuation arms 9 the annular element 10 moves along the longitudinal axis L towards the guiding element 5. Alternatively, the actuation unit may have an uneven number of arms 9. Generally, the number of actuation arms 9 is at least three.

The actuation arms 9 and the annular element 10 form in the shown embodiment a radial-to-axial linkage constructed and arranged to convert a radial movement provided by the actuation unit 9 to an axial movement of the annular element 10 and the shaft 4.

Further, in the shown embodiment, the annular element 10 of the driving unit 8 includes a flange 11 radially extending from the longitudinal axis L. The distal ends 9^{D} of the actuation arms 9 are pivotably connected to said flange 11 such that the actuation arms 9 are mutually coupled and move as an ensemble when at least a subset of actuation arms 9 is manually compressed.

As particularly shown in FIG. 2, the annular element 10 of the driving unit 8 is axially coupled to shaft 4. In the advantageous embodiment shown in FIG. 2, the annular element 10 is removably coupled to the shaft 4, using a bayonet coupling. Alternatively, another type or removable coupling can be used such as a snap connection. Also, the annular element 10 can be permanently coupled to the shaft 4.

The actuation arms 9 each include at least two arm segments 9′, 9″ and at least a hinge 9″′ interconnecting the at least two arm segments 9′, 9″, the arm segments 9′, 9″ having a stiffness that is larger than a stiffness of the hinge 9″′. By providing the actuation arms with portions having different stiffness, a bendable arm structure is realized. By pressing the arms radially inwardly, at least portions of the arms 9 bend radially inwardly. The arm segments 9′, 9″ can be made separately and assembled to each other, via the hinge 9″′, or, alternatively, can be made as an integral part having locally different stiffness values. In principle, further arm segments can be added to the two segment arm structure, e.g. via an additional hinge. A local stiffness can be set by making the arm thinner or thicker and/or by changing its material. As a further alternative, the actuation arm can be formed without hinge 9″′, however preferably such that the actuation arm bends upon manual actuation.

The actuation arms 9 can be designed in a way to provide different material properties to set the haptic conditions of the mechanism.

Further, the actuation arms 9 or basket can be personalized for instance as a 2k injected part with variable and different hardness and/or flexibility of the at least 2 arm segments 9′, 9″.

The actuation unit 9 further includes a proximal stationary element 13 that remains axially stationary relative to the handling unit 2. The arm proximal ends 9^{P} of the actuation arms are connected to the proximal stationary element 13, while the arm distal ends 9^{D} of the actuation arms 9 are connected to the flange 11 of the annular element 10, e.g. via respective intermediate hinges.

In the decompressed state, the annular element 10, or the flange 11 thereof, is located in a rest axial position R along the longitudinal axis L, as shown in FIG. 1. In the compressed state, the annular element 10, or the flange 11 thereof, is driven to an extended axial position E along the longitudinal axis L of the handling unit 2, also shown in FIG. 1, closer to the guiding element 5 than in the decompressed state.

A spring element (not shown) may be provided for biasing the annular element 11 towards the handling unit 2. Similarly, the actuation arms 9 are biased towards the decompressed state. Then, the actuation arms 9 are in the decompressed state or are at least tending towards said decompressed state, when not manually actuated.

In the shown embodiment, the guiding element 5 is stationary relative to the handling unit 2.

As discussed, the ensemble of the actuation unit 9 and the driving unit 8 are arranged for driving the shaft 4, in particular for driving the shaft 4 away from the handling unit 2 upon manual actuation of the actuation unit 9. Here, the surgery unit 3 may be axially stationary relative to the handling unit 2. It is noted however, that, in principle, the ensemble of the actuation unit 9 and the driving unit 8 may be arranged for moving the surgery unit 3 along the longitudinal axis L, while the shaft 4 remains axially stationary relative to the handling unit 2. Then, the driving unit 8 may be axially coupled, directly or indirectly, with the surgery unit 3.

In the shown exemplary embodiment, the guiding element 5 is provided with a nosecone 19 comprising at least one cone-shaped portion 20 or truncated cone-shaped portion having a proximal end 21 and a distal end 22, wherein the nosecone is located at a distal position with respect to the actuation arms 9.

In a particular embodiment, in a decompressed state, a distal end 9^{D} of at least one of the actuation arms 9 is located at a first axial distance A1 from the proximal end 21 of the cone-shaped part 20 of the nosecone 19 between about 8 mm and about 16 mm. Further, the distal end 9^{D} of at least one of the actuation arms 9 may be located at a second axial distance A2 from the distal end 22 of the cone-shaped part 20 of the nosecone 19 between about 12 mm and about 25 mm. Preferably, the cone-shaped portion 20 of the nosecone 19 has an axial length A of between about 2 mm and about 10 mm.

The surgical module 3 is formed as an integral part or as an assemblage of separate parts. The surgical module 3 may have a rod that is provided, at a distal end thereof, with an operating unit, such as a forceps, pair of scissors, fibre, scraper, vertical scissor or fiber assembly. At least one the actuation arms 9 of the actuation unit may have an outer or exterior contour deviating from an outer or exterior contour of other actuation arms, such that the at least one actuation arm is aligned with a circumferential orientation of the operating unit of the surgical module 3, e.g. the orientation of the forceps. By moving the shaft 4 relative to the surgical module 3, the distal end can selectively be exposed or opened so as to actuate or operate the operating unit, e.g. for grabbing and/or releasing tissue.

The invention is not restricted to the embodiments described herein. It will be understood that many variants are possible.

These and other embodiments will be apparent for the person skilled in the art and are considered to fall within the scope of the invention as defined in the following claims. For the purpose of clarity and a concise description features are described herein as part of the same or separate embodiments. However, it will be appreciated that the scope of the invention may include embodiments having combinations of all or some of the features described.

1. An ophthalmic surgery instrument, comprising:
a handling unit having a longitudinal axis,
a surgical module provided on the handling unit and extending away therefrom along the longitudinal axis thereof, and
a shaft at least partially surrounding the surgical module,
wherein the surgical module and the shaft are mutually movable relative along the longitudinal axis of the handling unit,
a guiding element arranged at a distal end of the handling unit, the guiding element being provided with a central bore that is traversed by the surgical module and the shaft extending from the handling unit outwardly along the longitudinal axis thereof,
wherein the handling unit comprises
an actuation unit comprising a plurality of actuation arms arranged around the longitudinal axis of the handling unit, the actuation arms each having a proximal end and a distal end, and
a driving unit arranged to, upon manual actuation of the actuation unit, drive the shaft relative to the guiding element back and forth through the central bore of the guiding element,
wherein the driving unit includes an annular element arranged at a proximal side of the guiding element so as to move, in association with the shaft, along the longitudinal axis, and
wherein the number of actuation arms is at least three, and wherein the distal ends of the actuation arms are pivotably connected to the annular element such that upon compressing at least a subset of the actuation arms the annular element moves along the longitudinal axis towards the guiding element.

2. An ophthalmic surgery instrument according to claim 1, wherein the actuation arms and the annular element form a radial-to-axial linkage constructed and arranged to convert a radial movement provided by the actuation unit to an axial movement of the shaft.

3. An ophthalmic surgery instrument according to claim 1, wherein the annular element of the driving unit includes a flange radially extending from the longitudinal axis, and wherein the distal ends of the actuation arms are pivotably connected to said flange.

4. An ophthalmic surgery instrument according to claim 1, wherein the annular element of the driving unit is axially coupled to shaft.

5. An ophthalmic surgery instrument according to claim 1, wherein the annular element of the driving unit is removably coupled to the shaft.

6. An ophthalmic surgery instrument according to claim 1, wherein the actuation arms are bendable.

7. An ophthalmic surgery instrument according to claim 1, wherein the actuation arms are adjustable between a compressed state and a decompressed state, wherein the actuation arms in the compressed state are closer to the longitudinal axis of the handling unit than in the decompressed state.

8. An ophthalmic surgery instrument according to claim 1, wherein the actuation arms are biased towards the decompressed state.

9. An ophthalmic surgery instrument according to claim 1, wherein the number of actuation arms is even, preferably four, six or eight.

10. An ophthalmic surgery instrument according to claim 1, wherein the actuation arm includes at least two arm segments and at least a hinge interconnecting the at least two arm segments, the arm segments having a stiffness that is larger than a stiffness of the hinge.

11. An ophthalmic surgery instrument according to claim 1, wherein, in the decompressed state, the annular element is located in a rest axial position along the longitudinal axis of the handling unit, and wherein, in the compressed state, the annular element is driven to an extended axial position along the longitudinal axis of the handling unit, closer to the guiding element than in the decompressed state.

12. An ophthalmic surgery instrument according to claim 1, wherein the actuation unit further includes a proximal stationary element, and wherein the arm proximal ends are connected to the proximal stationary element.

13. An ophthalmic surgery instrument according to claim 1, wherein the guiding element is substantially axially stationary relative to the handling unit.

14. An ophthalmic surgery instrument according to claim 1, wherein the actuation unit and the driving unit are arranged for driving the shaft, in particular for driving the shaft away from the handling unit upon manual actuation of the actuation unit.

15. An ophthalmic surgery instrument according to claim 1, wherein the guiding element is provided with a nosecone comprising at least one cone-shaped portion having a proximal end and a distal end, wherein the nosecone is located at a distal position with respect to the actuation arms.

16. An ophthalmic surgery instrument according claim 15, wherein a distal end of at least one of the actuation arms is located at an axial distance from the proximal end of the cone-shaped part of the nosecone between about 8 mm and about 16 mm and/or at an axial distance from the distal end of the cone-shaped part of the nosecone between about 12 mm and about 25 mm.

17. An ophthalmic surgery instrument according to claim 15, wherein the cone-shaped portion of the nosecone has an axial length of between about 2 mm and about 10 mm.
